(19) United States
(12) Patent Application Publication
(10) Pub. No.: US 2008/0177268 A1
(43) Pub. Date: Jul. 24, 2008

(54) Minimally-Invasive Approach to Bone-Obstructed Soft Tissue

(76) Inventors: Wolfgang Daum, Groton, MA (US); Axel Winkel, Schwerin (DE); Thomas Gunther, Weimar (DE); Andreas Klink, Berlin (DE)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 12/035,312

(22) Filed: Feb. 21, 2008

Related U.S. Application Data
(63) Continuation of application No. 10/366,832, filed on Feb. 14, 2003, now abandoned.
(60) Provisional application No. 60/357,208, filed on Feb. 14, 2002.

(51) Int. Cl. A61B 17/16

(52) U.S. Cl. 606/80

(57) ABSTRACT

The subject invention pertains to a method and apparatus for placing a minimally-invasive access with respect to a patient's bone or other non-soft tissue. The subject invention can use a drilling machine incorporating an ultrasound motor. The subject drilling machine can be applied to sample, for example, bone biopsies under MRI control. In a specific embodiment, the subject ultrasound motor can be completely manufactured of non-magnetic materials, such as plastics, titanium, and titanium alloy, or ceramics and piezoceramics. The subject drilling apparatus can be placed into an MRI near field without influencing the image quality, and without the drilling apparatus itself being disturbed by the MRI magnet, gradient, or high-frequency field. The subject invention can incorporate good shielding with the subject drilling apparatus use of these materials, and can achieve minimal, if any, image distortions or so-called artifacts. Thus, the subject invention can involve the problem by use of non-magnetic materials of low magnetic susceptibility for the design of an actuation unit.

### CROSS-REFERENCE TO RELATED APPLICATIONS

The present application is a continuation of U.S. patent application Ser. No. 10/366,832, filed Feb. 14, 2003, which claims the benefit of U.S. Provisional Patent Application Ser. No. 60/357,208, filed Feb. 14, 2002, both of which are hereby incorporated by reference herein in their entirety, including any figures, tables, or drawings.

### BACKGROUND OF THE INVENTION

MRI devices provide excellent soft tissue images. It is therefore reasonable to perform interventions or surgical procedures in soft tissue under MRI control. Several soft tissue regions, however, such as the brain, spinal duct, or parts of the intervertebral disks, are covered by bones in a way that one has to bore through these obstacles. However, an MRI compatible drill-operation device is not yet known until today. The invention presented herein aims at placing a minimally-invasive access into such regions by means of an actuation unit based on an ultrasound motor.

### BRIEF SUMMARY OF INVENTION

The subject invention pertains to a method and apparatus for placing a minimally-invasive access with respect to a patient's bone or other non-soft tissue. The subject invention can use a drilling machine incorporating an ultrasound motor. The subject drilling machine can be applied to sample, for example, bone biopsies under MRI control. In a specific embodiment, the subject ultrasound motor can be completely manufactured of non-magnetic materials, such as plastics, titanium, and titanium alloy, or ceramics and piezoceramics. The subject drilling apparatus can be placed into an MRI near field without influencing the image quality, and without the drilling apparatus itself being disturbed by the MRI magnet, gradient, or high-frequency field.

The subject invention can incorporate good shielding with the subject drilling apparatus use of these materials, and can achieve minimal, if any, image distortions or so-called artifacts. Thus, the subject invention can involve the problem by use of non-magnetic materials of low magnetic susceptibility for the design of an actuation unit.

### BRIEF DESCRIPTION OF THE DRAWINGS

The invention presented herein shall be described in detail with the following figures:

FIG. 1 shows a specific embodiment of the subject drilling apparatus.

FIG. 2 shows a modular mimic display of a specific embodiment of the subject actuation unit

FIG. 3 shows an exploded view of a specific embodiment of the subject MRI compatible actuation unit.

FIG. 4 shows a specific embodiment of a control unit which can be utilized in conjunction with the subject actuation unit.

FIG. 5 shows a specific embodiment of the subject driving unit, with clamped spike or Kirschner wire.

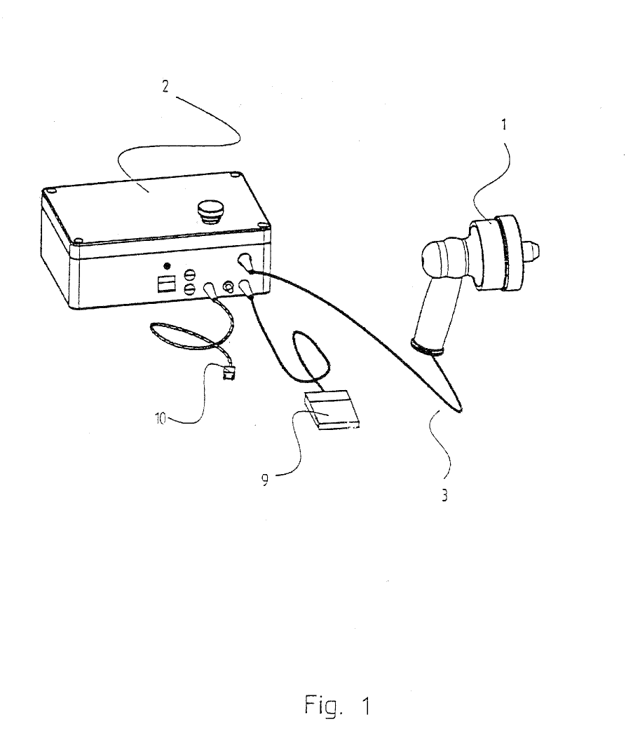

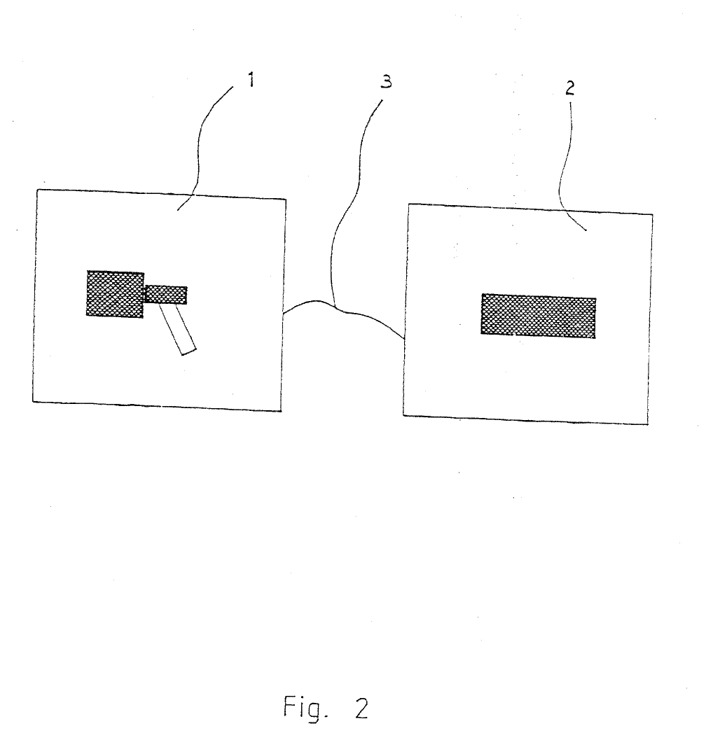

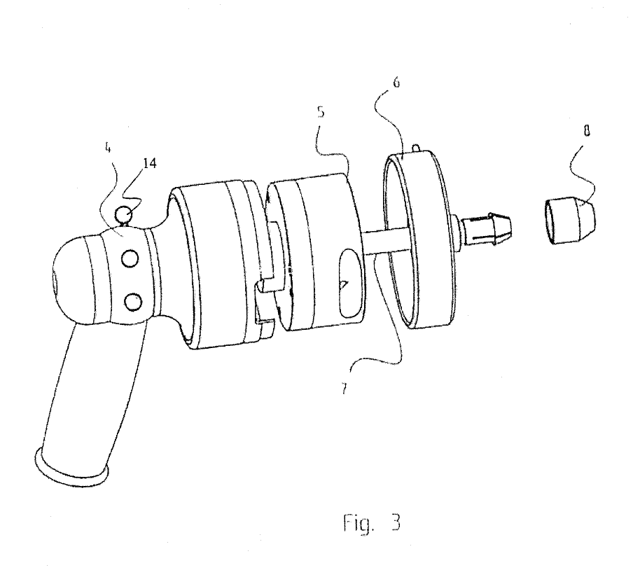

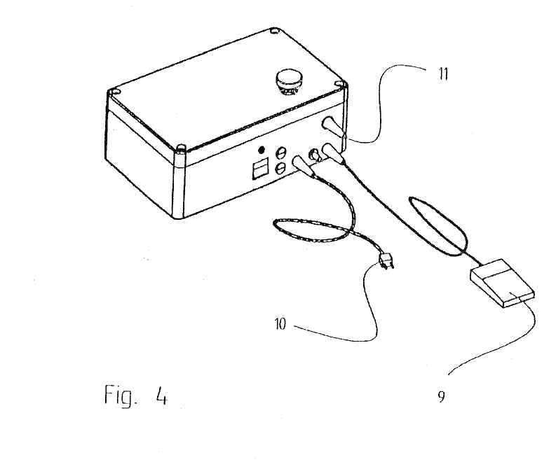

The subject actuation unit can incorporate a control unit 2 and the driving unit 1 (see FIGS. 1 and 2). The control unit 2 and driving unit 1 can be electrically connected by a cable 3. This connection can be separable by, for example, a pin- and socket connector 11 (see FIG. 4). A foot pedal 9 can be used to trigger the driving unit 1 during operation and can be connected to the control unit 2. In a specific embodiment, the driving unit 1 can incorporate case top 4, case underpart 6, for example hollow shaft 12, and piezomotor 5. Drilling chucks 8 can be used to ensure the seat of drills, for example with various diameters. FIG. 3 shows an exploded view of a specific embodiment of the subject activation unit, which is MRI compatible. In a specific embodiment, the subject driving unit 1 can be dismantled by the user, in order to remove the piezomotor 5 prior to sterilization. Thus, in a specific embodiment all parts of the Driving Unit 1, except for the piezomotor 5, can be sterilized.

### DETAILED DESCRIPTION OF THE INVENTION

The transformation of electrical energy into mechanical energy can occur by piezoelectric effect, eliminating a magnetic field that is typically produced by conventional electric motors. Additionally, the use of non-magnetic materials can produce an MRI image with minimal distortion.

The subject driving unit can incorporate a hollow shaft through which an instrument can pass and can be applied to the bone or other tissue being drilled. In a specific embodiment, the driving unit can be separated from the hollow shaft, allowing the hollow shaft to remain in the drilled hole and serve as minimally-invasive access to the respective tissue region.

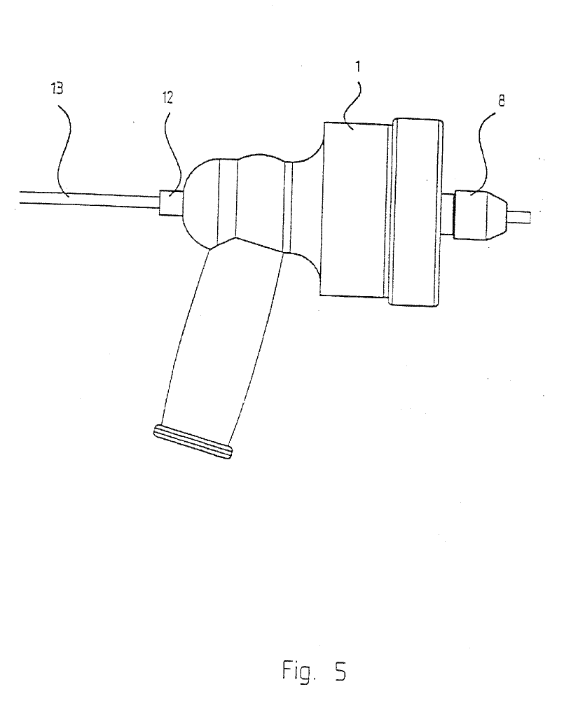

The subject invention can also allow the application of so-called spike and Kirschner wires 13. As FIG. 5 shows, those wires can be clamped into the hollow shaft 12 and applied for the respective borings into the chosen tissue areas. Subsequently, the wire clamping can be detached, and the driving unit 1 can be removed together with the hollow shaft 12. The wire 13 can then remain inside the tissue and serve as a marking. A hollow drill can then be clamped into the driving unit 1 to perform the required boring into the tissue along the wire 13.

It is possible to design the driving unit with markers that can be identified by a navigation system and allow the orientation of the device by means of such navigation system. The markers can be designed as reflectors or active optical light-emitting diodes. For application under MRI, the markers can be manufactured of a material which is actively or passively depictable under MRI. In a specific embodiment, the markers can be filled with a signal-emitting liquid. Such markers can be, for example, put onto the device or be integrated into the device structure.

### NAMES

1. Driving Unit
2. Control Unit
3. Electric cable
4. Case top with handle
5. Piezomotor
6. Case underpart
7. Clamping sleeve
8. Drilling chuck
9. Foot pedal
10. Mains cable
11. Pin-and-socket connector
12. Hollow shaft
13. Kirschner wire

1. A device for surgical and interventional application, comprising:
a driving unit, wherein the driving unit comprises
a motor, wherein the motor is substantially invisible under magnetic resonance imaging, wherein the motor comprises a hollow shaft, wherein the hollow shaft allows passage of an instrument through the motor to a patient's tissue.

2. The device according to claim 1, wherein the device is adapted for use in the neuro and spinal region, for orthopaedic interventions and trepanations.

3. The device according to claim 1, wherein the motor comprises external parts which can be sterilized and internal parts which cannot be sterilized wherein the device can be dismantled into the external parts of the device which can be sterilized and the internal parts of the device which cannot be sterilized.

4. The device according to claim 3, wherein the external parts comprise a case top and a case underpart, wherein the case top and case underpart can be sterilized via superheated steam or by plasma sterilization.

5. The device according to claim 1, further comprising a control unit wherein the control unit is connected to the driving unit via a cabling wherein the control unit controls the operation of the driving unit.

6. The device according to claim 5, wherein the driving unit is manufactured from materials creating small susceptibility artifacts within an MRI device.

7. The device according to claim 5, wherein the motor and the cabling are shielded in a way that image interference caused by the electric field of the motor and disturbances of the motor function induced by the electromagnetic MRI field are minimized.

8. The device according to claim 1, wherein instruments can be inserted and clamped through the hollow shaft.

9. The device according to claim 8, wherein Kirschner wires can be inserted and clamped through the hollow shaft.

10. The device according to claim 1, wherein the motor is an ultrasound motor, wherein the hollow shaft of the ultrasound motor allows the application of instruments.

11. The device according to claim 1, wherein the driving unit is separable from the hollow shaft, such that the hollow shaft can be left as a defined minimally-invasive access to a certain tissue area.

12. The device according to claim 11, wherein the hollow shaft can be left as a defined minimally-invasive access to the spinal region.

13. The device according to claim 1, further comprising markers, wherein the device can be connected to a navigation system which can detect the markers.

14. The device according to claim 1, further comprising:
a hollow drill clamped into the driving unit, wherein an instrument passing through the motor passes through the hollow drill to a patient's tissue.
